(19) United States
(12) Patent Application Publication
(10) Pub. No.: US 2003/0224039 A1
(43) Pub. Date: Dec. 4, 2003

(54) Methods for entrapment of bioactive agent in a liposome or lipid complex

(75) Inventors: Lawrence T. Boni, Monmouth Junction, NJ (US); Brian Miller, Mercerville, NJ (US); Fangjun Wu, Livingston, NJ (US)

Correspondence Address: ALLEN BLOOM, C/O DECHERT, PRINCETON PIKE CORPORATION CENTER, P.O. BOX 5218, PRINCETON, NJ, 08543-5218, US

(73) Assignee: Transave, Inc., Monmouth Junction, NJ

(21) Appl. No.: 10/383,004

(22) Filed: Mar. 5, 2003

Related U.S. Application Data
(60) Provisional application No. 60/361,809, filed on Mar. 5, 2002.

(51) Int. Cl. A61K 31/704; A61K 31/65; A61K 31/4709; A61K 9/127

(52) U.S. Cl. 424/450; 514/37; 514/312; 514/152

(57) ABSTRACT

A method of preparing a liposomal bioactive agent comprising infusing an lipid-ethanol mixture with an aqueous or ethanolic solution of the bioactive agent at a temperature below the phase transition of at least one of the lipid components of the lipid and compositions produced by the method of the invention.

The present application claims the benefit of the priority of U.S. Provisional Patent Application No. 60/361,809 filed Mar. 5, 2002, the disclosure of which is hereby incorporated by reference as if fully set forth herein.

The present invention relates to methods of entrapment of bioactive agents in a liposome or lipid complex. It is known in the art that entrapment of an bioactive agent in a liposome or lipid complex must be performed at a temperature higher than the phase transition of the lipid component with the highest melting point. The present invention comprises a method of entrapment of an bioactive agent in a liposome or lipid complex at a temperature lower than the phase transition of at least one of the lipid components. Surprisingly this method has demonstrated success and results in a high entrapment of the bioactive agent.

An obstacle to known methods of manufacture of liposomal antibacterial agents is that the processes utilize water immiscible or toxic solvents. In addition the size of the resultant vesicles is difficult to adjust. There are well-established methods for generation of liposomes greater than 1 micron and methods to then homogenize them to less than 0.03 microns. The intermediate range is more difficult to produce.

The method of manufacture of the present invention does not utilize either water immiscible or toxic solvents. The process is simple and scalable. Small unilamellar vesicles or lipids can be sterile filtered for aseptic processing. The size of vesicle formed can be adjusted without extrusion by varying the lipid composition, lipid concentrations, excipients, temperature, and shearing forces. Furthermore the size of the vesicles is intermediate which is generally preferable to the size of vesicles manufactured by other processes.

### BRIEF DESCRIPTIONS OF THE INVENTION

The present invention is directed to a method of entrapment of a bioactive agent in a liposome or lipid complex comprising infusing an lipid-ethanol mixture with the bioactive agent at a temperature below the phase transition of at least one of the lipid components of the lipid mixture.

In one embodiment the method of entrapment of a bioactive agent in a liposome or lipid complex comprises:

a) preparing an aqueous or ethanolic solution containing the bioactive agent;

b) preparing an lipid-ethanol solution; and,

c) infusing the lipid-ethanol solution into the aqueous or ethanolic solution containing the bioactive agent to produce a product. The step of infusing is performed at a temperature below the phase transition of at least one of the lipid components of the lipid-ethanol solution. The temperature can preferably be below 40 degres Celsius, below 35 degrees Celsius, or below 20 degrees Celsius. Further, the method can comprise the step of washing the product, preferably by dialysis or diafiltration.

The concentration of the lipid-ethanol solution is preferably below approximately 50 mg/mL and more preferably below approximately 30 mg/mL.

The step of infusing the lipid-ethanol solution into the aqueous or ethanolic solution containing the bioactive agent can be performed above or below the surface of the aqueous or ethanolic solution containing the bioactive agent. Preferably the step is performed above the surface of the solution.

Dialysis is performed in the presence of NaCl or Na₂SO₄, preferably with a concentration of between approximately 1.5% w/v and 3.0% w/v.

The aqueous or ethanolic solution containing the bioactive agent can contain a buffer.

In another embodiment the method of entrapment of a bioactive agent in a liposome or lipid complex comprises the steps of:

a) preparing an aqueous or ethanolic solution containing the bioactive agent;

b) preparing small unilamellar vesicles;

c) mixing the aqueous or ethanolic solution containing the bioactive agent with the small unilamellar vesicles to make a resultant solution,

d) infusing ethanol into the resultant solution to produce a product. The step of infusing is performed at a temperature below the phase transition of at least one of the lipid components of the lipid-ethanol solution. The step may be performed at a temperature between approximately 10 degrees Celsius and approximately 40 degrees Celsius. The method can further comprise the step of washing the product which may be achieved by dialysis or diafiltration.

The present invention also relates to a composition adapted for intravenous administration or inhalation comprising a liposomal bioactive agent produced by the process of the invention.

### BRIEF DESCRIPTION OF THE FIGURES

FIG. 1: Diagram of a preferred embodiment of a method of entrapment of the present invention.

FIG. 2: Diagram of a preferred embodiment of a method of entrapment of the present invention.

FIG. 3: Graphical representation of comparative lipid/drug ratio for varying lipid concentrations

FIG. 4: Graphical comparison of entrapment for various medii of dialysis.

FIG. 5 is a graphical representation of amikacin/lipid ratio compared with amount of DOPC.

FIG. 6 is a graphical representation of vesicle size compared with amount of DOPC.

FIG. 7 is a graphical representation of kill area compared with amount of DOPC.

FIG. 8 is a graphical representation of amikacin/lipid ratio compared with amount of cholesterol.

FIG. 9 is a graphical representation of vesicle size compared with amount of cholesterol.

FIG. 10 is a graphical representation of kill area compared with amount of cholesterol.

### DETAILED DESCRIPTION OF THE INVENTIONS

The term “bioactive agent” or “agent” is used throughout the specification to describe a compound or composition with biological activity. Bioactive agents of the present invention include agents which can be used for the treatment and prevention of conditions in a number of therapeutic areas. These therapeutic areas include: infectious disease (anti-bacterial, anti-fungal and anti-viral activity, vaccines,), inflammatory disease (including arthritis, and hypertension), neoplastic disease, diabetes, osteoporosis, pain management, general cardiovascular disease and lung disease. Lung disease includes: asthma, emphysema, lung cancer, chronic obstructive pulmonary disease (COPD), bronchitis, influenza, pneumonia, tuberculosis, respiratory distress syndrome, cystic fibrosis, sudden infant death syndrome (SDKs), respiratory synctial virus (RSV), AIDS related lung diseases (e.g., Pneumocystis carinii pneumonia, Mycobacterium.avium.complex, fungal infections, etc.), sarcoidosis, sleep apnea, acute respiratory distress syndrome (ARDS), bronchiectasis, bronchiolitis, bronchopulmonary dysplasia, coccidioidomycosis, hantavirus pulmonary syndrome, histoplasmosis, pertussis and pulmonary hypertension a biologically active agent which acts to kill or inhibit the growth of certain other harmful or pathogenic organisms, including, but not limited to bacteria, yeast, viruses, protozoa or parasites and which can be administered to living organisms, especially animals such as mammals, particularly humans. The term “bioactive agent” also includes compounds or compositions used for gene therapy and imaging.

Some specific examples of bioactive agents that can be encapsulated using methods of the present invention include: sulfonamide, such as sulfonamide, sulfamethoxazole and sulfacetamide; trimethoprim, particularly in combination with sulfamethoxazole; a quinoline such as norfloxacin and ciprofloxacin; a beta-lactam compound including a penicillin such as penicillin G, penicillin V, ampicillin, amoxicillin, and piperacillin, a cephalosporin such as cephalosporin C, cephalothin, cefoxitin and ceftazidime, other beta-lactam antibacterial agents such as imipenem, and aztreonam; a beta lactamase inhibitor such as clavulanic acid; an aminoglycoside such as gentamycin, amikacin, erthyromycin, tobramycin, neomycin, kanamycin and netilmicin; a tetracycine such as chlortetracycline and doxycycline; chloramphenicol; a macrolide such as erythromycin; or miscellaneous antibacterial agents such as clindamycin, a polymyxin, and bacitracin for anti-bacterial, and in some cases antifungal, infections; a polyene antibacterial agent such as amphotericin B, nystatin, and hamycin; flucytosine; an imidazole or a triazole such as ketoconazole, miconazole, itraconazole and fluconazole; griseofulvin for anti-Fungal diseases such as aspergillosis, candidaisis or histoplasmosis; zidovudine, acyclovir, ganciclovir, vidarabine, idoxuridine, trifluridine, an interferon (e.g, interferon alpha-2a or interferon alpha-2b) and ribavirin for anti-viral disease; aspirin, phenylbutazone, phenacetin, acetaminophen, ibuprofen, indomethacin, sulindac, piroxicam, diclofenac; gold and steroidal anti-inflammatories for inflammatory diseases such as arthritis; an ACE inhibitor such as captopril, enalapril, and lisinopril; the organo nitrates such as amyl nitrite, nitroglycerin and isosorbide dinitrate; the calcium channel blockers such as diltiazem, nifedipine and verapamil; the beta adrenegic antagonists such as propranolol for cardiovascular disease; a diuretic such as a thiazide; e.g., benzothiadiazine or a loop diuretic such as furosemide; a sympatholytic agent such as methyldopa, clonidine, gunabenz, guanaethidine and reserpine; a vasodilator such as hydalazine and minoxidil; a calcium channel blocker such as verapimil; an ACE inhibitor such as captopril for the treatment of hypertension; quinidine, procainamide, lidocaine, encainide, propranolol, esmolol, bretylium, verapimil and diltiazem for the treatment of cardiac arrhythmia; lovostatin, lipitor, clofibrate, cholestryamine, probucol, and nicotinic acid for the treatment of hypolipoproteinemias; an anthracycline such as doxorubicin, daunorubicin and idarubicin; a covalent DNA binding compound, a covalent DNA binding compound and a platinum compound such as cisplatin and carboplatin; a folate antagonist such as methotrexate and trimetrexate; an antimetabolite and a pyrimidine antagonist such as fluorouracil, 5-fluorouracil and fluorodeoxyuridine; an antimetabolite and a purine antagonist such as mercaptopurine, 6-mercaptopurine and thioguanine; an antimetabolite and a sugar modified analog such as cytarabine and fludarabine; an antimetabolite and a ribonucleotide reductase inhibitor such as hydoxyurea; a covalent DNA binding compound and a nitrogen mustard compound such as cyclophosphamide and ifosfamide; a covalent DNA binding compound and an alkane sulfonate such as busulfane; a nitrosourea such as carmustine; a covalent DNA binding compound and a methylating agent such as procarbazine; a covalent DNA binding compound and an aziridine such as mitomycin; a non covalent DNA binding compound; a non covalent DNA binding compound such as mitoxantrone and, bleomycin; an inhibitor of chromatin function and a topoisomerase inhibitor such as etoposide, teniposide, camptothecin and topotecan; an inhibitor of chromatin function and a microtubule inhibitor such as the vinca alkaloids including vincristine, vinblastin, vindisine, and paclitaxel, taxotere or another taxane; a compound affecting endocrine function such as prednisone, prednisolone, tamoxifen, leuprolide, ethinyl estradiol, an antibody such as herceptin; a gene such as the p-53 gene, the p 16 gene, the MIT gene, and the gene E-cadherin; a cytokine such as the interleukins, particularly, IL-1, IL-2, IL-4, IL-6, IL-8 and IL-12, the tumor necrosis factors such as tumor necrosis factor-alpha and tumor necrosis factor-beta, the colony stimulating factors such as granulocyte colony stimulating factor (G-CSF), macrophage colony stimulating factor (M-CSF) and, granulocyte macrophage colony stimulating factor (GM-CSF) an interferon such as interferon-alpha, interferon-beta 1, interferon-beta 2, and interferon-gamma; all-trans retinoic acid or another retinoid for the treatment of cancer; an immunosupressive agent such as: cyclosporine, an immune globulin, and sulfasazine, methoxsalen and thalidoimide; insulin and glucogon for diabetes; calcitonin and sodium alendronate for treatment of osteoporosis, hypercalcemia and Paget's Disease; morphine and related opioids; meperidine or a congener; methadone or a congener; an opioid antagonist such as nalorphine; a centrally active antitussive agent such as dexthromethrophan; tetrahydrocannabinol or marinol, lidocaine and bupivicaine for pain management; chloropromazine, prochlorperazine; a cannabinoid such as tetrahydrocannabinol, a butyrophenone such as droperidol; a benzamide such as metoclopramide for the treatment of nausea and vomiting; heparin, coumarin, streptokinase, tissue plasminogen activator factor (t-PA) as anticoagulant, antithrombolytic or antiplatelet drugs; heparin, sulfasalazine, nicotine and adrenocortical steroids and tumor necrosis factor-alpha for the treatment of inflammatory bowel disease; nicotine for the treatment of smoking addiction; growth hormone, luetinizing hormone, corticotropin, and somatotropin for hormonal therapy; and adrenaline for general anaphylaxis.

The term “liposomal” is used throughout the application to describe an agent which is encapsulated in or associated with a liposome or lipid complex. A lipid complex is an agent which is associated with one or more lipids.

The term “treatment” or “treating” means administering a composition to an animal such as a mammal or human for preventing, ameliorating, treating or improving a medical condition.

Liposomal bioactive agents can be designed to have a sustained therapeutic effect or lower toxicity allowing less frequent administration and an enhanced therapeutic index. Liposomes are composed of bilayers that entrap the desired pharmaceutical. These can be configured as multilamellar vesicles of concentric bilayers with the pharmaceutical trapped within either the lipid of the different layers or the aqueous space between the layers.

The lipids used in the compositions of the present invention can be synthetic, semi-synthetic or naturally-occurring lipids, including phospholipids, tocopherols, steroids, fatty acids, glycoproteins such as albumin, negatively-charged lipids and cationic lipids. Phosholipids include egg phosphatidylcholine (EPC), egg phosphatidylglycerol (EPG), egg phosphatidylinositol (EPI), egg phosphatidylserine (EPS), phosphatidylethanolamine (EPE), and egg phosphatidic acid (EPA); the soya counterparts, soy phosphatidylcholine (SPC); SPG, SPS, SPI, SPE, and SPA; the hydrogenated egg and soya counterparts (e.g., HEPC, HSPC), other phospholipids made up of ester linkages of fatty acids in the 2 and 3 of glycerol positions containing chains of 12 to 26 carbon atoms and different head groups in the 1 position of glycerol that include choline, glycerol, inositol, serine, ethanolamine, as well as the corresponding phosphatidic acids. The chains on these fatty acids can be saturated or unsaturated, and the phospholipid can be made up of fatty acids of different chain lengths and different degrees of unsaturation. In particular, the compositions of the formulations can include dipalmitoylphosphatidylcholine (DPPC), a major constituent of naturally-occurring lung surfactant as well as dioleoylphosphatidylcholine (DOPC) and dioleoylphosphatidylglycerol (DOPG). Other examples include dimyristoylphosphatidycholine (DMPC) and dimyristoylphosphatidylglycerol (DMPG) dipalmitoylphosphatidcholine (DPPC) and dipalmitoylphosphatidylglycerol (DPPG) distearoylphosphatidylcholine (DSPC) and distearoylphosphatidylglycerol (DSPG), dioleylphosphatidylethanolamine (DOPE) and mixed phospholipids like palmitoylstearoylphosphatidylcholine (PSPC) and palmitoylstearoylphosphatidylglycerol (PSPG), and single acylated phospholipids like mono-oleoyl-phosphatidylethanolamine (MOPE).

In a preferred embodiment the lipid employed is a saturated phosphatidycholine with a well defined phase transition, such as DPPC.

The lipid-ethanol solution used can comprise dipalmitoylphosphatidylcholine (DPPC), dioleoylphosphatidylcholine (DOPC), cholesterol and dioleoylphosphatidylglycerol (DOPG). The molar ratio of DPPC:DOPC:cholesterol:DOPG may be 59:5:30:6. The lipid-ethanol solution may comprise dipalmitoylphosphatidylcholine (DPPC) and cholesterol in a molar ratio of 1:1.

The entrapment decreases as the amount of DOPC is increased above 30% DOPC. A similar trend is observed in the biological activity of these liposomes. As observed by light microscopy the formation of sheets occurs above about 80% DPPC.

The process demonstrates a decreased mol to mol lipid to bioactive agent ratio when compared with known processes. More specifically the lipid to bioactive agent ratio using the process of the present invention is less than 5 to 1. More preferably the lipid to bioactive agent ratio using the process of the present invention is less than 3 to 1. Still more preferably the lipid to bioactive agent ratio is less than 2.5 to 1.

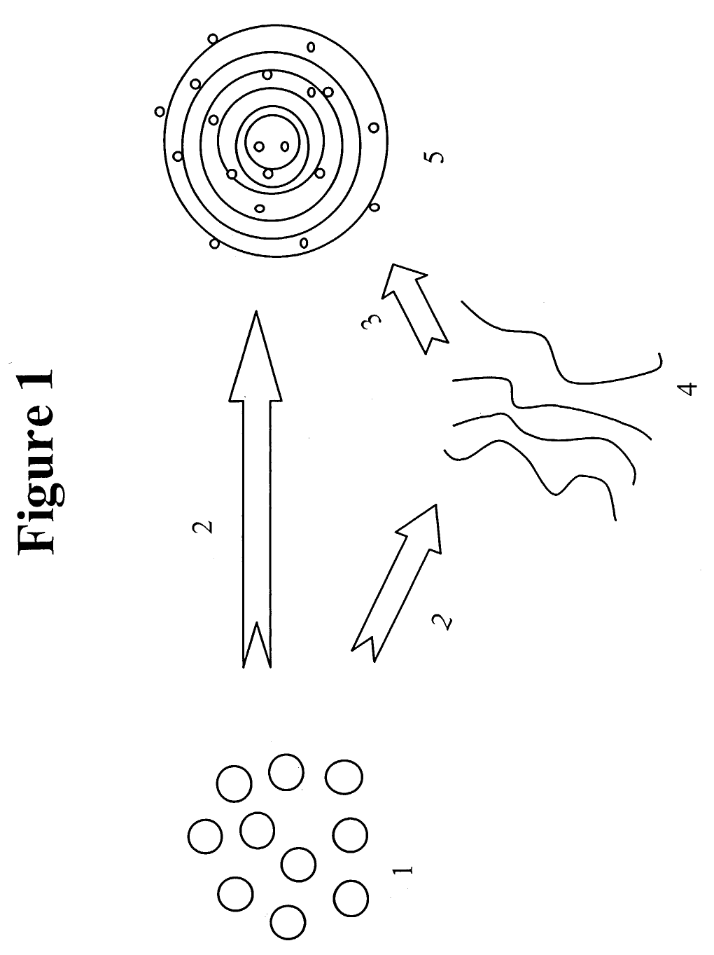

An embodiment of the process of manufacture of the present invention is shown in FIG. 1. Liposomes (1) in the form of small unilamellar vesicles (SUVs) are mixed with an aqueous or ethanolic solution (2) containing the bioactive agent to be entrapped. Ethanol is infused into this mixture. The mixture immediately forms either extended sheets of lipid (3) or multilamellar vesicles (MLVs).(5) The extended sheets of lipid will form MLVs upon removal of ethanol (4) by either sparging or washing by such methods as centrifugation, dialysis or diafiltration. The MLVs will range in diameter between approximately 0.1 and approximately 3.0 μm.

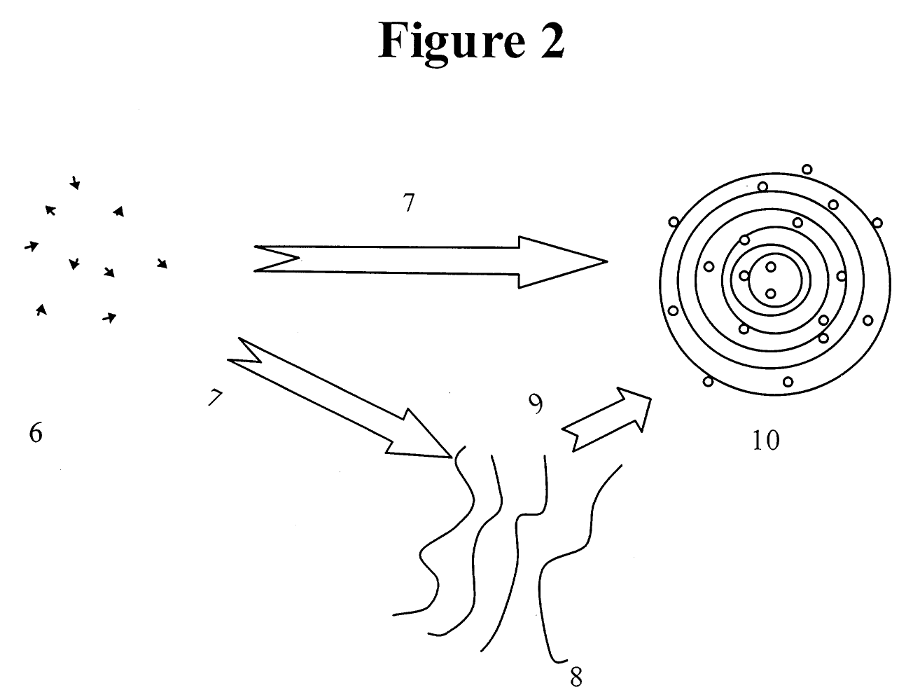

A second embodiment is shown in FIG. 2. The lipids to be employed are dissolved in ethanol to form a lipid-ethanol solution (6). The lipid-ethanol solution is infused in an aqueous or ethanolic solution containing the molecule of the bioactive agent to be entrapped (7). All manipulations are performed below the phase transition of the lowest melting lipid. The mixture immediately forms either extended sheets of lipid (8) or multilamellar vesicles (MLVs).(10) The extended sheets of lipid will form MLVs upon removal of ethanol (9) by either sparging or washing by such methods as centrifugation, dialysis or diafiltration. The MLVs will range in diameter from approximately 0.1 to approximately 3.0 μm.

In a preferred embodiment of the invention the concentration of the lipid ethanol solution is less than 50 mg/mL. In a more preferred embodiment the concentration of the lipid-ethanol solution is less than 30 mg/mL.

In another preferred embodiment dialysis is performed using NaCl solution with a concentration of between approximately 0.5% w/v and approximately 3.5%w/v. In a more preferred embodiment dialysis is performed using Na₂SO₄ solution with a concentration of between approximately 0.5% w/v and approximately 3.5%w/v. In an even more preferred embodiment dialysis is performed using Na₂SO₄ solution with a concentration of between approximately 1.5% w/v and approximately 3.0%w/v

In a preferred embodiment ethanol is infused into the aqueous or ethanolic solution containing the bioactive agent from above the surface of the solution.

For the entrapment of lipophilic molecules the molecules are first dissolved in ethanol with the lipids and this mixture is infused into the aqueous phase.

The process can be easily adapted for large scale, aseptic manufacture. The final liposome size can be adjusted by modifying the lipid composition, concentration, excipients, and processing parameters. Without limiting the scope of the application it is believed that the slow sealing of the vesicles may be responsible for the high level of entrapment.

Table 1 compares one embodiment of the method of entrapment of the present invention with known methods of entrapment. The table compares the lipid to drug ratio and the size of the resultant vesicles. The method of the present invention (E) demonstrates a lower lipid to drug ratio and smaller vesicle size.

A. 1 mL stock lipid-ethanol solution was dried on a rotovaporator to produce 30 mg lipid. 3.23 mL stock amikacin was added at 50 degrees Celsius.

B. 1 mL stock lipid-ethanol solution was dried on a rotovaporator to produce 30 mg lipid. 3.23 mL stock amikacin was added. The solution was subjected to five cycles of freezing using dry ice/ethanol and thawing in a 50 degree Celsius bath.

C. 1 mL stock lipid-ethanol solution was dried on a rotovaporator to produce 30 mg lipid. 0.646 mL MeCl₂ were added. 3.23 mL amikacin solution were added. Gaseous N₂ was used to remove the MeCl₂.

D. 1 mL stock lipid-ethanol solution was infused with 3.23 mL amikacin solution at 50 degrees Celsius.

E. 1 mL stock lipid-ethanol solution was infused with 3.23 mL amikacin solution at 25 degrees Celsius.

| Sample | Free | | Total | |
|---|---|---|---|---|
| ID | [Amk] mg/ml | % free | lipid/Total drug | size, um |
| A:MLV | 0.070 | 22.9 | 19.9 | 1.28 |
| B, FATMLV | 0.055 | 19.6 | 23.3 | 1.16 |
| C, SPLV | 0.506 | 33.2 | 6.9 | 0.71 |
| D, ETOHINF. | 0.051 | 15.0 | 17.1 | 0.60 |
| E. | 0.042 | 2.8 | 3.3 | 0.58 |

### EXAMPLE 1

### Process for Encapsulating Amikacin

7.47 g DPPC and 3.93 g cholesterol were dissolved directly in 352.5 mL ethanol in a 50 C water bath. 85.95 g amikacin sulfate was dissolved directly in 1147.5 mL PBS buffer. The 57.3 mg/ml amikacin sulfate solution was then titrated with 10M NaOH or KOH to bring the pH to approximately 6.8.

352.5 mL of a 32.3 mg/ml ethanol/lipid solution was added or infused to the 1147.5 mL amikacin/buffer to give a total initial volume of 1.5 L. The ethanol/lipid was pumped at 30 mL/min (also called infusion rate) with a peristaltic pump into the amikacin/buffer solution which was being rapidly stirred at 150 RPM in a reaction vessel on a stir plate at 25 degrees Celsius

The product was stirred at 25 degrees Celsius for 20-30 minutes.

The mixing vessel was hooked up to a peristaltic pump and diafiltration cartridge. The diafiltration cartridge is a hollow membrane fiber with a molecular weight cut-off of 500 kilodaltons. The product was pumped from the reaction vessel through the diafiltration cartridge and then back into the mixing vessel at 25 degrees Celsius. A back pressure of approximately 7 psi is created throughout the cartridge. Free amikacin and ethanol were forced through the hollow fiber membrane by the back pressure leaving the liposomal amikacin (product) behind. The product was washed 8 times at 25 degrees Celsius. Fresh PBS buffer was added (via another peristaltic pump) to the reaction vessel to compensate for the permeate removal and to keep a constant product volume. The product was concentrated. 150 mL of liposomal amikacin were produced.

### EXAMPLE 1a

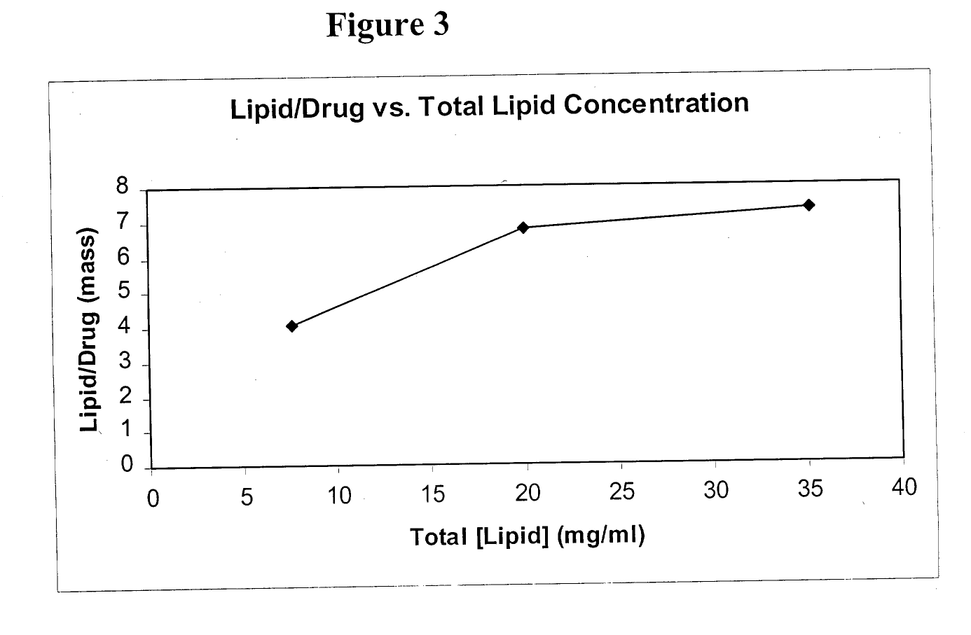

The process was repeated using 20.0 mg/mL lipid/ethanol solution and 35.2 mg/mL lipid ethanol solution. The lipid to drug ratio increased as the lipid solution concentration increased. (FIG. 3)

### EXAMPLE 1b

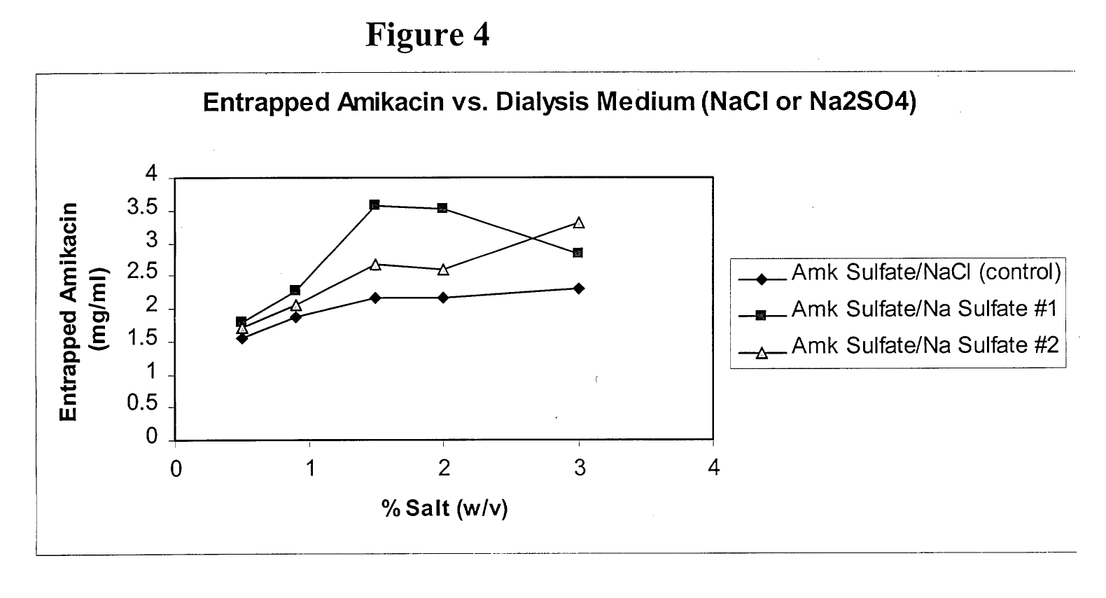

The process was repeated with dialysis performed using NaCl and Na₂SO₄ at varying concentrations. Lipid entrapment is best with a concentration of between approximately 1.5% w/v Na₂SO₄ and approximately 3% w/v Na₂SO₄. (FIG. 4)

### EXAMPLE 1c

The process was repeated using a 21.3 mg/mL lipid/ethanol solution. In the first case the ethanol was infused into the amikacin/buffer solution from above. In the second case the ethanol was infused directly into the amikacin/buffer solution from slightly below the surface of the solution. Entrapment was better when the ethanol was infused from above. (Table 2)

TABLE 2

| | | | Infusion | | | | | | | |
|---|---|---|---|---|---|---|---|---|---|---|
| | | Charged | Conditions | | Amikacin Base | | | Theoretical | Volume | |
| | Scale | Lipid | Mixing | Infusion | Total | Free % | % | Lipid/Drug | Ave. Size | |
| Batch # | (mL) | (mg/ml) | (RPM) | Position | (mg/ml) | (mg/ml) | Free | (mass) | (μm) | Comments |
| 5 | 300 | 5.0 | 150 | Above | 4.22 | 0.099 | 2.3 | 7.9 | 0.326 | Concentrated |
| | | | | Solution | | | | | (Res. = 25.3) | 6.7 fold |
| 6 | 300 | 5.0 | 150 | In | 3.26 | 0.061 | 1.9 | 10.3 | 0.223 | Concentrated |
| | | | | Solution | | | | | (χ² = 1.0) | 6.7 fold |
| | | | | Slightly | | | | | | |
| | | | | below | | | | | | |
| | | | | surface | | | | | | |

### EXAMPLE 1d

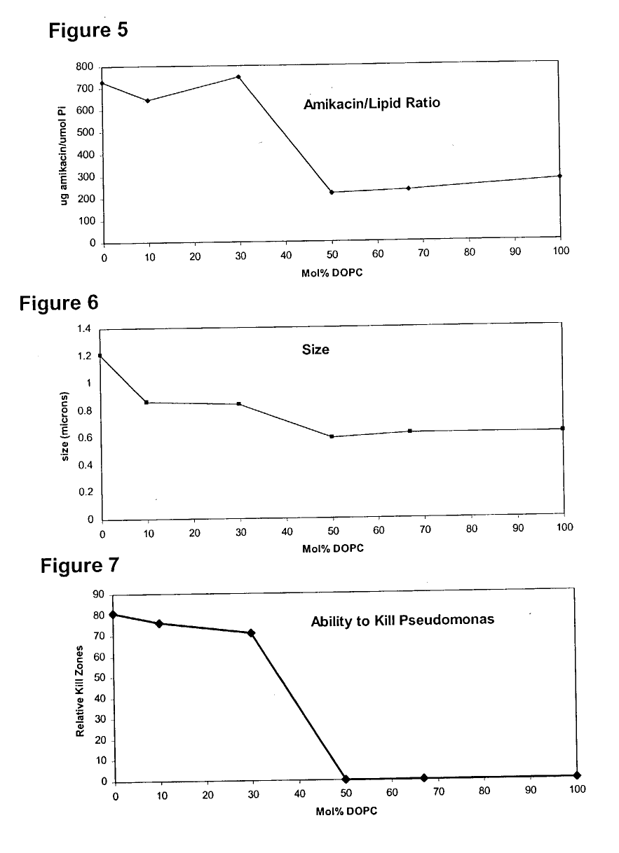

Mixtures of DPPC and DOPC at defined ratios are dissolved in ethanol. A stock solution of lipid for each ratio is made at 32.5 mglipid/ml. The molecule to be entrapped is amikacin sulfate made at a stock of 75 mg/ml in 10 mM Hepes buffer, pH 6.8, 150 mM NaCl. To 1 ml amikacin stock infuse 0.31 ml ethanol/lipid stock solution at room temperature. The results are given in FIGS. 5, 6 and 7.

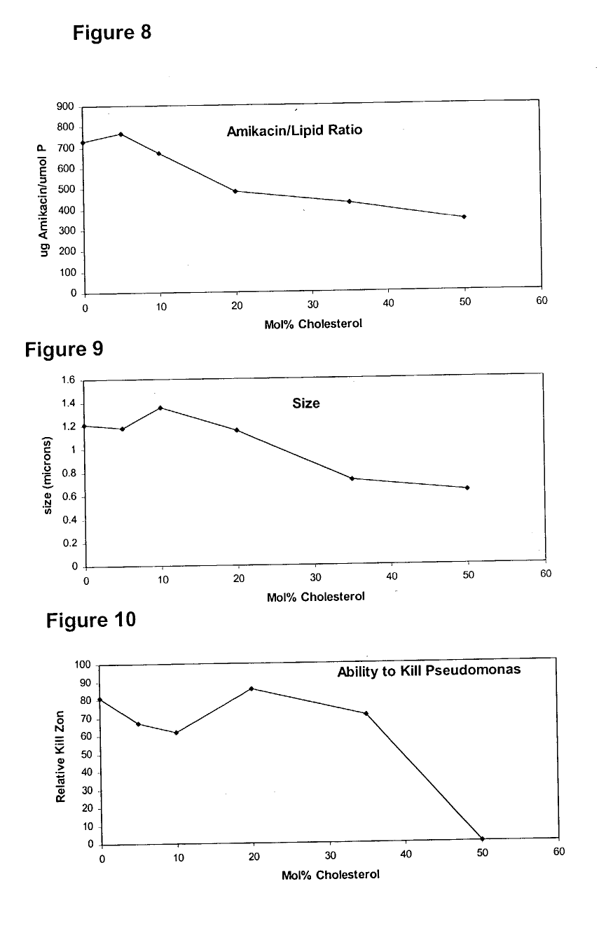

Performed as above with cholesterol in place of DOPC. Sheets were observed upon infusion at 90% DPPC. At 80% a mixture of sheets and vesicles was present. The results are given in FIGS. 8, 9 and 10.

### EXAMPLE 2

### Process for encapsulating ciprofloxacin

141.7 mg DPPC and 8.3 mg cholesterol were dissolved in chloroform, then rotoevaporated and left overnight on a vacuum to remove the chloroform. The resulting thin film was then hydrated with 1.5 mL of citrate buffer at pH 5 to give a 100 mg/ml MLV solution. The MLV solution was then sonicated until SUVs were formed (1 hour). A 16 mg/ml stock ciprofloxacin solution in citrate buffer at pH 5 was prepared. These were mixed as follows.

At 25 degrees Celsius 0.764 mL SUV (100 mg/ml) was added to 0.764(16 mg/ml Cipro Stock) and 0.470 mL EtOH to produce a 2 mL sample volume. The sample was then dialyzed in citrate buffer at pH 5.

### EXAMPLE 3

### Process for encapsulating gentamicin

DPPC/DOPC/Chol./DOPG (59/5/30/6 mol ratio) were dissolved in ethanol to produce a 32.3 mg/mL lipid-ethanol solution.

A 75 mg/ml gentimicin sulfate solution was titrated with 10M NaOH or KOH to bring the pH to approximately 6.8.

35.3 mL of the 32.3 mg/mL ethanol-lipid solution was infused to 114.7 mL gentimicin sulfate solution in 10 mM Hepes. The ethanol/lipid was pumped at 30 mL/min (also called infusion rate) with a peristaltic pump into the gentimicin/buffer solution which was being rapidly stirred at 150 RPM in a reaction vessel on a stir plate at 25 degrees Celsius

The product was stirred at 25 degrees Celsius for 20-30 minutes before diafiltration with NaCl. Final entrapment after washing by diafiltration was Lipid/drug mass ratio of 7.8.

We claim:

1. A method of preparing a liposomal bioactive agent comprising infusing an lipid-ethanol mixture with an aqueous or ethanolic solution of the bioactive agent at a temperature below the phase transition of at least one of the lipid components of the lipid.

2. The method of claim 1 wherein the bioactive agent is an antibacterial agent.

3. The method of claim 2 wherein the antibacterial agent is an aminoglycoside.

4. The method of claim 3, wherein the aminoglycoside is amikacin.

5. The method of claim 2 wherein the antibacterial agent is a quinoline.

6. The method of claim 3 wherein the aminoglycoside is a tetracycline.

7. The method of claim 1 wherein the ratio of bioactive agent to lipid is less than approximately 3:1.

8. The method of claim 1 wherein the ratio of bioactive agent to lipid is less than approximately 2.5:1

9. The method of claim 17, wherein the temperature is below approximately 40 degrees Celsius.

10. The method of claim 17, wherein the temperature is below approximately 30 degrees Celsius.

11. The method of claim 1 wherein the concentration of the lipid-ethanol solution is below approximately 50 mg/mL.

12. A method of entrapment of a bioactive agent in a liposome or lipid complex comprising the steps of:
a) preparing an aqueous or ethanolic solution containing the bioactive agent;
b) preparing an lipid-ethanol solution; and,
c) infusing the lipid-ethanol solution into the aqueous or ethanolic solution containing the bioactive agent to produce a product, wherein the step of infusing is performed at a temperature below the phase transition of at least one of the lipid components of the lipid-ethanol solution.

13. The method of claim 12 further comprising the step of washing the product.

14. The method of claim 13, wherein the step of washing the product comprises dialysis or diafiltration.

15. The method of claim 12 wherein the concentration of the lipid-ethanol solution is below approximately 50 mg/mL.

16. The method of claim 12 wherein the concentration of the lipid-ethanol solution is below approximately 30 mg/mL.

17. The method of claim 12 wherein the step of infusing the lipid-ethanol solution into the aqueous or ethanolic solution containing the bioactive agent is performed above the surface of the aqueous or ethanolic solution containing the bioactive agent.

18. The method of claim 14, wherein the dialysis is performed in the presence of NaCl.

19. The method of claim 14, wherein the dialysis is performed in the presence of Na₂SO₄

20. The method of claim 19, wherein the Na₂SO₄ has a concentration of between approximately 1.5% w/v and 3.0% w/v.

21. The method of claim 12 wherein the bioactive agent is an antibacterial agent.

22. The method of claim 21, wherein the antibacterial agent is an aminoglycoside.

23. The method of claim 21, wherein the antibacterial agent is amikacin.

24. The method of claim 21, wherein the antibacterial agent is gentimicin

25. The method of claim 21, wherein the antibacterial agent is ciprofloxacin.

26. The method of claim 12 wherein the aqueous or ethanolic solution containing the bioactive agent further contains a buffer.

27. A method of entrapment of a bioactive agent in a liposome or lipid complex comprising the steps of:
a) preparing a aqueous or ethanolic solution containing the bioactive agent;
b) preparing small unilamellar vesicles;
c) mixing the aqueous or ethanolic solution containing the bioactive agent with the small unilamellar vesicles to make a resultant solution,
d) infusing ethanol into the resultant solution to produce a product, wherein the step of infusing is performed at a temperature below the phase transition of at least one of the lipid components of the lipid-ethanol solution.

28. The method of claim 27 further comprising the step of washing the product.

29. The method of claim 28, wherein the step of washing the product comprises dialysis or diafiltration.

30. The method of claim 27 wherein the step of infusing the ethanol into the resultant solution is performed above the surface of the resultant solution.

31. The method of claim 29, wherein the dialysis is performed in the presence of NaCl

32. The method of claim 29, wherein the dialysis is performed in the presence of Na₂SO₄

33. The method of claim 32, wherein the Na₂SO₄ has a concentration of between approximately 1.5% w/v and 3.0% w/v.

34. The method of claim 27 wherein the bioactive agent is an antibacterial agent.

35. The method of claim 34, wherein the antibacterial agent is an aminoglycoside.

36. The method of claim 34, wherein the antibacterial agent is amikacin.

37. The method of claim 34, wherein the antibacterial agent is gentimicin

38. The method of claim 34, wherein the antibacterial agent is ciprofloxacin.

39. The method of claim 27 wherein the aqueous or ethanolic solution containing the bioactive agent further contains a buffer.

40. A composition adapted for intravenous administration comprising a liposomal bioactive agent produced by the process of claim 1.

41. A composition adapted for administration by inhalation comprising a liposomal bioactive agent produced by the process of claim 1.

42. A composition adapted for intravenous administration comprising a liposomal bioactive agent produced by the process of claim 12.

43. A composition adapted for administration by inhalation comprising a liposomal bioactive agent produced by the process of claim 12.

44. A composition adapted for intravenous administration comprising a liposomal bioactive agent produced by the process of claim 27.

45. A composition adapted for administration by inhalation comprising a liposomal bioactive agent produced by the process of claim 27.

46. The method of claim 12, wherein the lipid-ethanol solution comprises dipalmitoylphosphatidylcholine (DPPC), dioleoylphosphatidylcholine (DOPC), cholesterol and dioleoylphosphatidylglycerol (DOPG).

47. The method of claim 46, wherein the molar ratio of DPPC:DOPC:cholesterol:DOPG is 59:5:30:6.

48. The method of claim 12, wherein the lipid-ethanol solution comprises dipalmitoylphosphatidylcholine (DPPC), dioleoylphosphatidylcholine (DOPC), cholesterol and dioleoylphosphatidylglycerol (DOPG) and the bioactive agent is gentamicin.

49. The method of claim 48, wherein the molar ratio of DPPC:DOPC:cholesterol:DOPG is 59:5:30:6.

50. The method of claim 12, wherein the lipid-ethanol solution comprises dipalmitoylphosphatidylcholine (DPPC) and cholesterol.

51. The method of claim 46, wherein the molar ratio of DPPC:cholesterol is 1:1.

52. The method of claim 12, wherein the lipid-ethanol solution comprises dipalmitoylphosphatidylcholine (DPPC) and cholesterol and the bioactive agent is amikacin.

53. The method of claim 52, wherein the molar ratio of DPPC:cholesterol is 1:1.
